(19) United States
(12) Patent Application Publication
(10) Pub. No.: US 2006/0184171 A1
(43) Pub. Date: Aug. 17, 2006

(54) Flexible element for use in a stabilization device for bones or vertebrae

(76) Inventors: Lutz Biedermann, VS-Villingen (DE); Wilfried Matthis, Weisweil (DE); Jurgen Harms, Karlsruhe (DE)

Correspondence Address: CHRISTIE, PARKER & HALE, LLP, PO BOX 7068, PASADENA, CA, 91109-7068, US

(21) Appl. No.: 11/274,449

(22) Filed: Nov. 15, 2005

Related U.S. Application Data
(60) Provisional application No. 60/628,811, filed on Nov. 17, 2004.

(30) Foreign Application Priority Data
Nov. 17, 2004 (DE) 10 2004 055 454.4

(51) Int. Cl. A61F 2/30

(52) U.S. Cl. 606/61

(57) ABSTRACT

A flexible element is provided for use in a stabilization device for bones or vertebrae. The flexible element comprises a flexible section arranged between a first end and a second end. The flexible section has curved sections that alternatingly extend away from opposite sides of a connecting axis that extends from the first end through the flexible section and the second end.

### CROSS-REFERENCE TO RELATED APPLICATION

This application claims the benefit of U.S. Provisional Application No. 60/628,811, filed Nov. 17, 2004.

### FIELD OF THE INVENTION

The present invention relates to a flexible element for use in a stabilization device for bones or vertebrae that comprises a flexible section.

### BACKGROUND OF THE INVENTION

Fixation and stabilization devices are commonly used to fix bone fractures or to stabilize a spinal column. These fixation and stabilization devices typically consist of at least two bone anchoring elements, which are each anchored in a bone or vertebra. The bone anchoring elements are connected by a rigid plate or rod and do not permit any motion of the bones or vertebrae relative to each other.

In some instances, however, a dynamic stabilization of the bones or vertebrae is desirable wherein the bones and vertebrae are allowed to move with a controlled limited motion relative to each other. Dynamic stabilization can be achieved, for example, by using a flexible element instead of a rigid plate or rod to connect the bone anchoring elements.

For example, U.S. Patent Application Publication No. 2003/0191470 A1 teaches a flexible element for connecting bone anchoring elements consisting of a rod with a center section having a curve that extends to one side of the rod axis. The center section thereby exerts a restoring force when the rod is deflected from a resting position. Because the curve extends to only one side of the rod axis, however, the flexible element comprises an asymmetric shape and locally high loads act on the rod.

In addition, U.S. Pat. No. 6,440,169 B1 teaches a flexible element for the stabilization of vertebrae consisting of two leaf springs. The leaf springs, however, only allow a limited compressive motion in a direction of the connection axis of the vertebrae.

Further, U.S. Patent Application Publication No. 2003/0220643 A1 teaches a rod for connecting bone anchoring elements consisting of a flexible portion formed in the shape of a substantially helical spring. The flexural strength of the flexible portion is the same in all directions perpendicular to the rod axis and, therefore, no directed flexural strength is given.

### BRIEF SUMMARY OF THE INVENTION

It is therefore an object of the invention to provide a flexible element having a direction-dependent flexural strength perpendicular to a rod axis and high strength under cyclical load, which is capable of being easily varied for use with a wide variety of stabilization devices for vertebrae or bones and/or selectively combined with a wide variety of stabilization devices for vertebrae or bones.

This and other objects are achieved by a flexible element for use in a stabilization device for bones or vertebrae comprising a rod extending between a first end and a second end. The rod has curved sections that alternatingly extend away from opposite sides of a connecting axis that extends from the first end through the rod and the second end.

This and other objects are further achieved by a flexible element for use in a stabilization device for bones or vertebrae comprising a flexible section arranged between a first end and a second end. The flexible section has curved sections that alternatingly extend away from opposite sides of a connecting axis that extends from the first end through the flexible section and the second end. The curved sections have a teardrop shape.

This and other objects are still further achieved by a flexible element for use in a stabilization device for bones or vertebrae comprising a first end and a second end and a flexible section that extends from the first end to the second end. The flexible section has curved sections that alternatingly extend away from opposite sides of a connecting axis that extends from the first end through the flexible section and the second end. The flexible section has a substantially S-shape when viewed in a direction perpendicular to the connecting axis.

### BRIEF DESCRIPTION OF THE DRAWINGS

FIG. 1 is a perspective view of a flexible element according to a first embodiment;

FIG. 2 is a side view of the flexible element according to the first embodiment;

FIG. 3 is an enlarged perspective view of a section of the flexible element according to the first embodiment;

FIG. 4 is a perspective view of a flexible element according to a second embodiment;

FIG. 5 is a side view of the flexible element according to the second embodiment;

FIG. 6 is a perspective view of a flexible element according to a third embodiment;

FIG. 7 is a side view of the flexible element according to the third embodiment;

FIG. 8 is a perspective view of a flexible element according to a fourth embodiment; and

FIG. 9 is a partial sectional schematic illustration of the flexible element according to the first embodiment being used in a stabilization device.

### DETAILED DESCRIPTION OF THE INVENTION

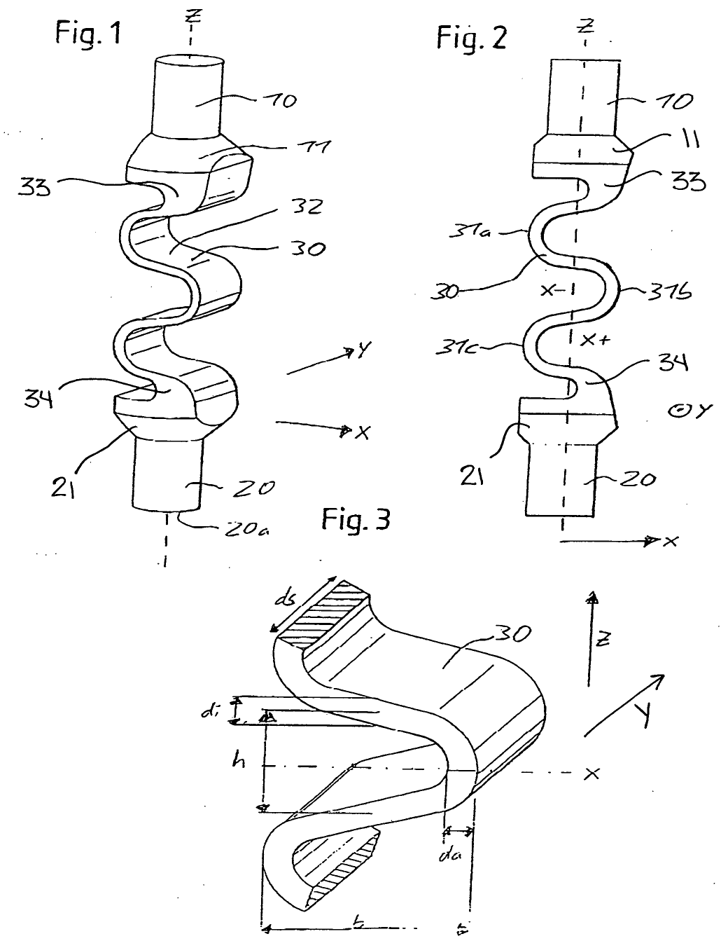

FIGS. 1-3 show a flexible element according to a first embodiment of the invention. As shown in FIGS. 1-2, the flexible element has a first end 10, a second end 20, and a flexible section 30 arranged there between. The first end 10, the second end 20, and the flexible section 30 are formed in one piece. The flexible element may be made, for example, of a biocompatible material, such as titanium. Alternatively, the flexible element may be made, for example, of a biocompatible shape memory alloy having superelasticity, such as Nickel Titanium Naval Ordnance Laboratory (NITINOL).

The first end 10 and the second end 20 each have a substantially cylindrical cross-section having an axes arranged substantially parallel to a connecting axis Z of the first end 10, the flexible section 30, and the second end 20. A first conical section 11 joins the first end 10 to the flexible section 30. The first conical section 11 conically widens from the first end 10 to the flexible section 30. A second conical section 21 joins the second end 20 to the flexible section 30. The second conical section 21 conically widens from the second end 20 to the flexible section 30.

The flexible section 30 is a substantially flat rod 32 having a substantially rectangular cross-section. As shown in FIG. 2, the flat rod 32 is formed into a substantially sinuous shape to have a plurality of curved sections 31a, 31b, 31c. The substantially sinuous shape extends from the first conical section 11 to the second conical section 21. The curved sections 31a, 31b, 31c extend in a direction X perpendicular to the connecting axis Z and alternate from a first side X+ to a second side X− of the connecting axis Z such that the flexible section 30 is asymmetrical with respect to the connecting axis Z. In the illustrated embodiment, the curved section 31b is positioned on the first side X+ of the connecting axis Z and the curved sections 31a, 31c are positioned on the second side X− of the connecting axis Z. Although three of the curved sections 31a, 31b, 31c are shown in the illustrated embodiment, it will be appreciated by those skilled in the art that the number of the curved sections 31 may be more or less than three depending on the desired properties of the flexible element.

The parameters of the flexible section 30 directly influence the flexural properties of the flexible element and can be adapted to obtain a desired result. As shown in FIG. 3, the flexible section 30 contains the following parameters: ds (width of the flexible section 30 in a direction Y perpendicular to the connecting axis Z and to the direction X), b (twice the amplitude of the wave), h (half of the wave length), da (thickness of the flexible section 30 in the direction X at the curved sections 31a, 31b, 31c), and di (thickness of the flexible section 30 in the direction of the connecting axis Z at the connecting axis Z).

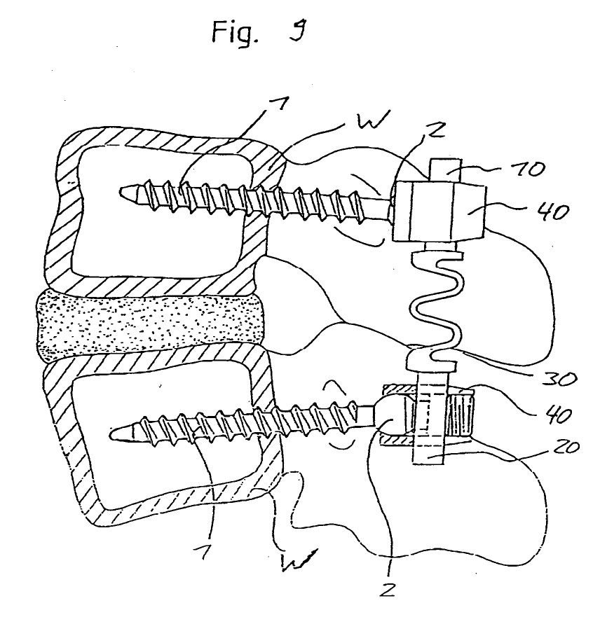

In the illustrated embodiment, the flexible section 30 has a constant width ds over its whole length in the direction Y. Additionally, when the flexible element is used, for example, in a stabilization device for bones or vertebrae (FIG. 9), the length of the first and second ends 10, 20 and the length of the flexible section 30 can be selected according to the distance between the bone anchoring elements and the required flexural properties of the flexible element.

Because the flexible section 30 is formed with the flat rod 32, which has a substantially sinuous shape, the flexible element has a high torsional strength with respect to torsion around the connecting axis Z and a high flexural strength with respect to flexural load in the direction Y (i.e., flexion around an axis extending in the direction X), a high elasticity with respect to a flexural load in the direction X (i.e., flexion around an axis extending in the direction Y), and a high elasticity with respect to compression and extension in the direction of the connecting axis Z. By increasing the parameter ds, the torsional strength and the flexural strength in the direction Y can be increased at the same time. Additionally, with the appropriate adjustment of the other parameters h, da, di and b, the flexural strength and the elastic spring deflection in the direction of the connecting axis Z can be systematically adjusted.

FIG. 9 shows the flexible element according to the first embodiment being used in a stabilization device. As shown in FIG. 9, the stabilization device comprises first and second bone anchoring elements, such as polyaxial bone screws. The polyaxial bone screws each have a shank 1 and a head 2. Each of the shanks 1 is anchored, for example, in a vertebra W of a spinal column. Each of the heads 2 are held in a receiving member 40 such that the heads 2 are pivotable and lockable in an angular position by a fixation element. The first end 10 and the second end 20 of the flexible element are each accommodated in one of the receiving members 40. Each of the polyaxial bone screws are thereby connected to the adjacent vertebrae W.

By using the flexible element in such an arrangement, a controlled motion of the vertebrae W relative to each other is enabled in that an elastic translatory motion in the direction of the connecting axis Z of the flexible element and an elastic flexural motion in the direction X are allowed, and a torsional motion and a flexural motion in the direction Y are largely prevented. Additionally, by appropriate selection of the parameters described with reference to FIG. 3, the desired properties of the flexible element with respect to the controlled motion can be easily adjusted and the flexible element can be easily varied for use with a wide variety of stabilization devices comprising, for example, monoaxial bone screws, polyaxial bone screws, rods, or plates. The flexible element can also be selectively combined with a wide variety of stabilization devices for vertebrae or bones.

The flexible element is also compact and at the same time has a direction-dependent flexural strength. This is particularly important when the flexible element is used in a spinal column, particularly a cervical spine, where the available space is considerably less than that in a lumbar region. Further, the shape of the flexible element can easily be changed so that a wide range of elastic properties can be attained. In addition, because the flexible section 30 has the curved sections 31a, 31b, 31c positioned on both sides of the connecting axis Z, the restoring force is substantially the same with respect to deflections in opposite directions from the resting position. As a result, the stress on the material of the flexible element is more evenly distributed under cyclical load compared to known flexible elements, which increases the life of the flexible element and reduces the danger of the material cracking due to fatigue. A bending stress which is almost constant over the mean length is also attained, and the dynamic axial deflection keeps the translatory motion acting at the facet joints level, which helps to prevent arthrosis at the facet joints.

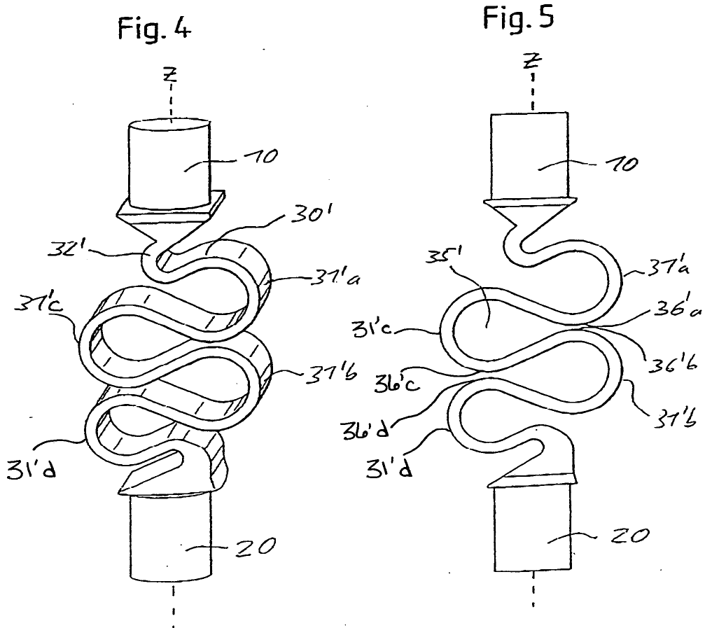

FIGS. 4-5 show a flexible element according to a second embodiment of the invention. Elements of the second embodiment that are identical to elements of the first embodiment will be described using the same reference numerals and will not be described in further detail. The second embodiment differs from the first embodiment in that the second embodiment has a flexible section 30′ formed from a substantially flat rod 32′. The flat rod 32′ has a substantially meandering shape formed to have a plurality of curved sections 31′a, 31′b, 31′c, 31′d. In a side view, the curved sections 31′a, 31′b, 31′c, 31′d have a larger diameter in open regions 35′ than at the connecting axis Z, which is unlike the curved sections 31a, 31b, 31c of the first embodiment, such that each of the curved sections 31′a, 31′b, 31′c, 31′d has a substantially teardrop shape that extends substantially perpendicular to the connecting axis Z. As shown in FIG. 5, side faces 36′a. 36′b, of adjacent curved sections 31′a, 31′b and side faces 36′c, 36′d of adjacent curved sections 31′c, 31′d are positioned proximate each other and spaced a smaller distance apart than side faces of the adjacent curved sections 31a, 31b of the first embodiment. In addition to the uses and advantages set forth with regard to the first embodiment, in the flexible element according to the second embodiment, elastic spring deflection in the direction of the connecting axis Z can be limited and at the same time, by appropriate variation of the other parameters shown in FIG. 3, the flexural strength of the flexible element can be adjusted to achieve a desired result.

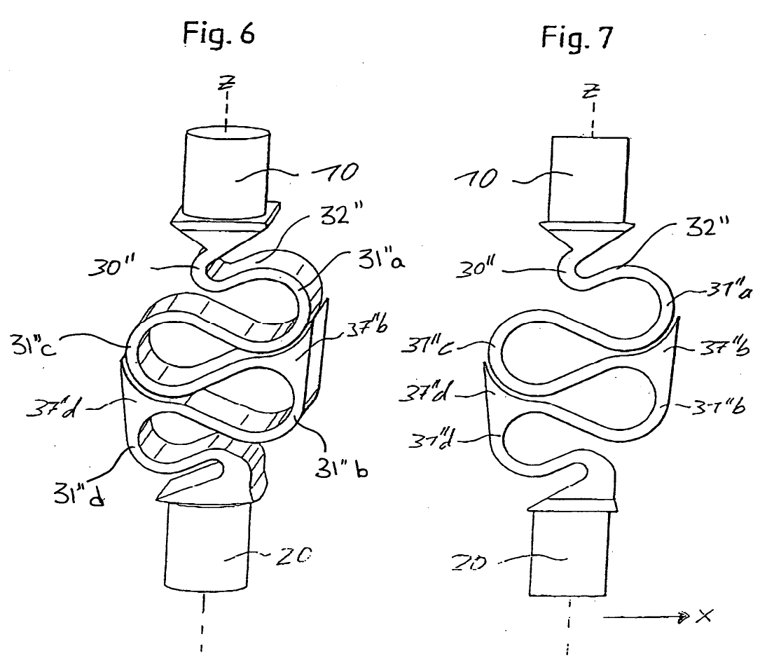

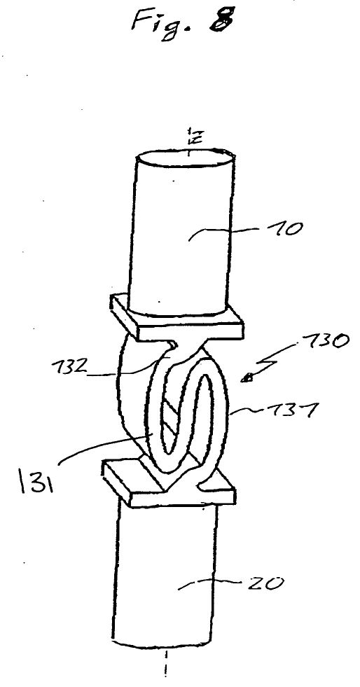

FIGS. 6-7 show a flexible element according to a third embodiment of the invention. Elements of the third embodiment that are identical to elements of the first and second embodiment will be described using the same reference numerals and will not be described in further detail. The third embodiment has a flexible section 30″ formed from a substantially flat rod 32″ having a plurality of curved sections 31″a, 31″b, 31″c, 31″d. The third embodiment differs from the second embodiment only in that the curved section 31″b of the third embodiment has an extension 37″b formed integrally therewith that extends toward the adjacent curved section 31″a, and on the opposite side of the connecting axis Z, the curved section 31″d of the third embodiment has an extension 37″d formed integrally therewith that extends toward the adjacent curved section 31″c. The extensions 37″b, 37″d are formed such that an interior side of the extension 37″b, 37″d facing the adjacent curved section 31″a, 31″c, respectively, substantially follows the shape of the respective adjacent curved section 31″a, 31″c and is positioned a small distance therefrom. An exterior side of the extension 37″b, 37″d extends along a connecting line from the curved section 31″b, 31″d to the adjacent curved section 31″a and 31″c, respectively, without being connected thereto. In addition to the uses and advantages set forth with regard to the previous embodiments, in the flexible element according to the third embodiment, the spring deflection of the flexible element in the direction of the connecting axis Z and the flexural or translatory motion in the direction X can be restricted.

FIG. 8 shows a flexible element according to a fourth embodiment of the invention. Elements of the fourth embodiment that are identical to elements of the first embodiment will be described using the same reference numerals and will not be described in further detail. The fourth embodiment differs from the first embodiment in that the fourth embodiment has a flexible section 130 formed from a substantially flat rod 132. The flat rod 132 has a substantially meandering shape formed to have a plurality of curved sections 131. The curved sections 131 extend away from opposite sides of the connecting axis Z and are more pronounced than the curved sections 31′a, 31′b, 31′c, 31′d of the second embodiment such that the flat rod 132 has a substantially S-shape in a middle of the flexible section 130 when viewed in a direction perpendicular to the connecting axis Z and adjacent curved sections 131 are located side by side. In addition to the uses and advantages set forth with regard to the previous embodiments, the flexible section 130 of the flexible element according to the fourth embodiment has a length shorter than the flexible sections 30, 30′, 30″ of the previous embodiments so that a more compact construction is possible.

The foregoing illustrates some of the possibilities for practicing the invention. Many other embodiments are possible within the scope and spirit of the invention. For example, it is possible to modify the cross-sectional shape of the flexible section 30, 30′, 30″, 130 or to modify the cross-sectional shape of the flexible section 30, 30′, 30″, 130 in a direction of extension of the flat rod 32, 32′, 32″, 132. Also, the first and second ends 10, 20 may have a modified shape and do not have to be formed integrally with the flexible section 30, 30′, 30″, 130. Other cross-sectional shapes of the flat rod 32, 32′, 32″, 132, such as a rectangular cross-section having rounded edges, are also possible. Additionally, the flexible element according to the embodiments described herein may be used in any conventional stabilization device for bones or vertebrae and are not limited to use in the stabilization device shown in FIG. 9. It is, therefore, intended that the foregoing description be regarded as illustrative rather than limiting, and that the scope of the invention is given by the appended claims together with their full range of equivalents.

What is claimed is:

1. A flexible element for use in a stabilization device for bones or vertebrae, comprising:
a first end and a second end; and
a rod extending between the first end and the second end, the rod having curved sections that alternatingly extend away from opposite sides of a connecting axis that extends from the first end through the rod and the second end.

2. The flexible element according to claim 1, wherein the rod is substantially flat.

3. The flexible element according to claim 1, wherein the curved sections extend substantially perpendicular to the connecting axis.

4. The flexible element according to claim 1, wherein the rod is asymmetrical with respect to the connecting axis.

5. The flexible element according to claim 1, wherein the rod has a sinuous shape.

6. The flexible element according to claim 1, wherein at least one of the first and second ends has a substantially cylindrical cross-section.

7. The flexible element according to claim 1, wherein the flexible element is made from a biocompatible material.

8. The flexible element according to claim 7, wherein the flexible element is made from a shape memory alloy.

9. A flexible element for use in a stabilization device for bones or vertebrae, comprising:
a first end and a second end; and
a flexible section arranged between the first end and the second end, the flexible section having curved sections that alternatingly extend away from opposite sides of a connecting axis that extends from the first end through the flexible section and the second end, the curved sections having a teardrop shape.

10. The flexible element according to claim 9, wherein the flexible section is formed from a substantially flat rod.

11. The flexible element according to claim 9, wherein the curved sections extend substantially perpendicular to the connecting axis.

12. The flexible element according to claim 9, wherein the flexible section is asymmetrical with respect to the connecting axis.

13. The flexible element according to claim 9, wherein the curved sections positioned on the same side of the connecting axis have side faces positioned proximate each other.

14. The flexible element according to claim 9, wherein at least one of the curved sections has an extension extending there from that extends toward and is positioned adjacent to an adjacent curved section positioned on the same side of the connecting axis.

15. The flexible element according to claim 9, wherein the flexible section has a meandering shape.

16. The flexible element according to claim 9, wherein at least one of the first and second ends has a substantially cylindrical cross-section.

17. The flexible element according to claim 9, wherein the flexible element is made from a biocompatible material.

18. The flexible element according to claim 17, wherein the flexible element is made from a shape memory alloy.

19. A flexible element for use in a stabilization device for bones or vertebrae, comprising:
a first end and a second end; and
a flexible section extending from the first end to the second end, the flexible section having curved sections that alternatingly extend away from opposite sides of a connecting axis that extends from the first end through the flexible section and the second end, the flexible section having a substantially S-shape when viewed in a direction perpendicular to the connecting axis.

20. The flexible element according to claim 19, wherein the flexible section is formed from a substantially flat rod.

21. The flexible element according to claim 19, wherein the flexible section has a meandering shape.

22. The flexible element according to claim 19, wherein at least one of the first and second ends has a substantially cylindrical cross-section.

23. The flexible element according to claim 19, wherein the flexible element is made from a biocompatible material.

24. The flexible element according to claim 23, wherein the flexible element is made from a shape memory alloy.

25. A stabilization device for bones or vertebrae, comprising:
at least two receiving members;
at least two bone anchoring elements each being connected to one of the receiving elements;
a flexible element including a flexible section arranged between a first end and a second end, the first end and the second end each being accommodated in one of the receiving members; and
the flexible section having curved sections that alternatingly extend away from opposite sides of a connecting axis that extends from the first end through the flexible section and the second end, the curved sections having a teardrop shape.

26. The stabilization device according to claim 25, wherein the flexible section is formed from a substantially flat rod.

27. The stabilization device according to claim 25, wherein the curved sections extend substantially perpendicular to the connecting axis.

28. The stabilization device according to claim 25, wherein the curved sections positioned on the same side of the connecting axis have side faces positioned proximate each other.

29. The stabilization device according to claim 25, wherein the flexible section is asymmetrical with respect to the connecting axis.

30. The stabilization device according to claim 25, wherein at least one of the curved sections has an extension extending there from that extends toward and is positioned adjacent to an adjacent curved section positioned on the same side of the connecting axis.

31. The stabilization device according to claim 25, wherein the flexible section has a meandering shape.

32. The stabilization device according to claim 25, wherein at least one of the first and second ends has a substantially cylindrical cross-section.

33. The stabilization device according to claim 25, wherein the flexible element is made from a biocompatible material.

34. The stabilization device according to claim 33, wherein the flexible element is made from a shape memory alloy.

35. A stabilization device for bones or vertebrae, comprising:
at least two receiving members;
at least two bone anchoring elements each being connected to one of the receiving elements;
a flexible element including a flexible section arranged between a first end and a second end, the first end and the second end each being accommodated in one of the receiving members; and
the flexible section extending from the first end to the second end, the flexible section having curved sections that alternatingly extend away from opposite sides of a connecting axis that extends from the first end through the flexible section and the second end, the flexible section having a substantially S-shape when viewed in a direction perpendicular to the connecting axis.

36. The stabilization device according to claim 35, wherein the flexible section is formed from a substantially flat rod.

37. The stabilization device according to claim 35, wherein the flexible section has a meandering shape.

38. The stabilization device according to claim 35, wherein at least one of the first and second ends has a substantially cylindrical cross-section.

39. The stabilization device according to claim 35, wherein the flexible element is made from a biocompatible material.

40. The stabilization device according to claim 39, wherein the flexible element is made from a shape memory alloy.

41. A stabilization device for bones or vertebrae, comprising:
at least two receiving members;
at least two bone anchoring elements each being connected to one of the receiving elements;
a flexible element including a rod extending between a first end and a second end, the first end and the second end each being accommodated in one of the receiving members; and
the rod having curved sections that alternatingly extend away from opposite sides of a connecting axis that extends from the first end through the rod and the second end.

42. The stabilization device according to claim 41, wherein the rod is substantially flat.

43. The stabilization device according to claim 41, wherein the rod has a sinuous shape.

44. The stabilization device according to claim 41, wherein at least one of the first and second ends has a substantially cylindrical cross-section.

45. The stabilization device according to claim 41, wherein the rod is made from a biocompatible material.

46. The stabilization device according to claim 45, wherein the rod is made from a shape memory alloy.
